(19) United States
(12) Patent Application Publication
(10) Pub. No.: US 2025/0319089 A1
(43) Pub. Date: Oct. 16, 2025

(54) STABLE LYOPHILISATES COMPRISING 5,10-METHYLENE-(6R)-TETRAHYDROFOLIC ACID

(71) Applicant: Merck Patent GMBH, Darmstadt (DE)

(72) Inventors: Rudolf Moser, Schaffhausen (CH); Viola Groehn, Dachsen (CH); Thomas Ammann, Marthalen (CH); Jean-Pierre Knapp, Schaffhausen (CH); Marianne Svaerd, Veberoed (SE)

(21) Appl. No.: 18/869,153

(22) PCT Filed: Jun. 5, 2023
(86) PCT No.: PCT/EP2023/064973

§ 371 Date: Nov. 25, 2024

(30) Foreign Application Priority Data
Jun. 8, 2022 (EP) 22177930.9
Jun. 8, 2022 (EP) 22177931.7

(51) Int. Cl. A61K 31/519; A61K 9/19

(52) U.S. Cl. CPC A61K 31/519; A61K 9/19

(57) ABSTRACT

The present invention relates to stable lyophilizates comprising a high content of 5,10-methylene-(6R)-tetrahydrofolic acid, which compositions do not contain any extraneous stabilizers or further chemotherapeutic agents.

The present invention relates to stable lyophilizates comprising a high content of 5,10-methylene-(6R)-tetrahydrofolic acid, which compositions do not contain any extraneous stabilizers or further chemotherapeutic agents.

### BACKGROUND OF THE INVENTION

5,10-methylenetetrahydrofolic acid is known as a medicament used in combination with 5-fluorouracil (5-FU) in the treatment of solid tumors (Seley, K. L. Drugs 4 (1), 99, 2001). The active isomeric form 5,10-methylene-(6R)-tetrahydrofolic acid (referred to as 5,10-CH₂-(6R)-THF in the following), achieves its chemotherapeutic effect together with the base analogue and 5-FU metabolite 5-FdUMP by inhibiting the enzyme thymidylate synthase (TS). TS catalyses the conversion of deoxyuridylate (dUMP) to deoxythymidylate (dTMP), which is an essential building block for DNA synthesis. Deactivation of TS occurs by formation of a covalent, ternary inhibition complex between TS, the base analogue 5-FdUMP, and 5,10-CH₂-(6R)-THF.

An enhancement of the cytotoxic effect of 5-FU can be achieved by increasing the intracellular concentration of 5,10-CH₂-(6R)-THF, whereupon the stability of the ternary inhibition complex is increased. This causes direct inhibition of DNA synthesis and repair, which ultimately results in cell death and delay of tumor growth. In order to achieve high intracellular concentrations of 5,10-CH₂-(6R)-THF the application of respective stable, high content products is desired.

However, there are undesirable properties associated with 5,10-CH₂-(6R)-THF that limit its pharmaceutical use. For example, 5,10-CH₂-(6R)-THF is highly susceptible to oxidation and chemical degradation that results in insufficient stability and unfavourably high levels of impurities.

5,10-methylenetetrahydrofolic acid is an addition product of tetrahydrofolic acid and formaldehyde (see e.g. Poe, M. et al. Biochemistry 18 (24), 5527, 1979; Kallen, R. G. Methods in Enzymology 18B, 705, 1971) and is known for its extremely high sensitivity to oxidation by air as well as instability in neutral and/or acidic environments potentially leading to chemical degradation and/or hydrolysis (see e.g. Odin, E. et al., Cancer Investigation 16 (7), 447, 1998; Osborn, M. J. et al., J. Am. Chem. Soc. 82, 4921, 1960; Hawkes, J., and Villota, R. Food Sci. Nutr. 28, 439, 1989). Susceptibility to oxidation, chemical degradation, and insufficient stability of 5,10-CH₂-(6R)-THF is especially apparent in aqueous solution, or when the compound is present in its amorphous form where it has a large surface (e.g. in its pharmaceutical use form as a lyophilizate), or in re-dissolved form such as solutions for injection. It is well known that to be amenable for pharmaceutical use, the respective composition needs to fulfill several requirements including high chemical and isomeric stability, such that effective storage over an acceptable period of time can be achieved, without exhibiting a significant change in the composition's physicochemical characteristics, ease of handling and processing, etc.

Attempts to stabilize compositions of 5,10-methylenetetrahydrofolates included e.g. (i) rigorous exclusion of atmospheric oxygen by the use of special technical devices for the reconstitution of solid formulations and the injection of 5,10-methylenetetrahydrofolates in an air-free environment (see e.g. Odin, E. et al., Cancer Investigation 16 (7), 447, 1998; U.S. Pat. No. 4,564,054); (ii) addition of a reducing agent such as L(+)-ascorbic acid or salts thereof, reduced gamma-glutathione, beta-mercaptoethanol, thioglycerol, N-acetyl-L-cysteine, etc. as an antioxidant for the highly sensitive 5,10-methylenetetrahydrofolic acid and for tetrahydrofolic acid in particular; (iii) stabilization by means of cyclodextrin inclusion compounds (see e.g. EP 0 579 996); (iv) addition of citrate while adjusting the pH to a basic value (see e.g. EP 1 641 460); or (v) formation of various crystalline forms such as the sulfate salts (see e.g. EP 0 537 492) or hemisulfate salts (see e.g. EP 2 837 631).

Lyophilizates of 5,10-CH₂-(6R)-THF have as described hereinabove previously been prepared from aqueous solutions which contain—in addition to the active compound, i.e. 5,10-CH₂-(6R)-THF—also dicarboxylic acids and/or tricarboxylic acids such as citric acid and/or other stabilizers, see e.g. WO2019034673, US 2007/0099866 and U.S. Pat. No. 10,059,710 B2. Solutions disclosed therein for the purpose of preparing lyophilizates contain at most 2-3% by weight 5,10-CH₂-(6R)-THF.

However, neither lyophilizates containing dicarboxylic acids and/or tricarboxylic acids such as citric acid and/or other stabilizers, nor the crystalline salt forms of 5,10-methylenetetrahydrofolic acid are readily useful for pharmaceutical purposes due to their low aqueous solubility, and moreover the stabilized versions of 5,10-methylenetetrahydrofolic acid known in the art usually contain less than 50% of the active drug compound 5,10-CH₂-(6R)-THF due the dilution in the final dosage form by the stabilizing additives.

As an example, the company Adventrx Pharmaceuticals carried out stability studies on their drug candidate CoFactor®, i.e. the calcium salt of the diastereomer mixture 5,10-methylene-(6R,S)-tetrahydrofolic acid, which were disclosed i.a. in WO 2007/064968. The chemical stability of the diastereomer mixture 5,10-CH₂-(6R,S)-THF is assumed to be similar to the pure diastereomer 5,10-CH₂-(6R)-THF of the present invention. The study compared the stability of nonformulated 5,10-methylene-(6R,S)-tetrahydrofolic acid with 5,10-methylene-(6R,S)-tetrahydrofolic acid formulated with only trisodium citrate or formulated with both ascorbic acid and trisodium citrate; both of which compounds are well-known reducing agents (see FIG. 1).

Linear regression analysis of the stability profiles of the isolated lyophilizates showed that the degradation of 5,10-methylene-(6R,S)-tetrahydrofolic acid was linear over time (see FIG. 2). The degradation rate (slope of the best-fit line) for each formulation (re-constituted lyophilizate) demonstrated the following order, from fastest to slowest degradation rate: nonformulated>formulated with only trisodium citrate>formulated with both ascorbic acid and trisodium citrate (FIG. 2). Nonformulated 5,10-methylene-(6R,S)-tetrahydrofolic acid was thus found to lose 2.3% purity per hour, resulting in a purity of 84% after 7 hours, whereas formulations containing trisodium citrate+ascorbic acid had much higher stability, resulting in a purity of about 95% after 7 hours.

Moreover, the solutions disclosed in WO 2007/064968 for the purpose of preparing the most stable lyophilizates contain less than 5% by weight 5,10-methylene-(6R,S)-tetrahydrofolic acid, and the resulting lyophilizates contain less than 20% by weight 5,10-methylene-(6R,S)-tetrahydrofolic acid (see FIG. 3).

Additionally, stabilizers such as citric acid, used to prepare the most stable lyophilizates in WO 2007/064968, for example, have been linked to various undesired effects like e.g. QTc elongation (Laspina et al. Transfusion 42 (2002) p. 899, Toyoshima et al. Clinical Nutrition (2006) 25, 653-660), inducing hypocalcaemia (Payne et. Al. J. Physiol. (1964), 170, pp. 613-620), etc. From a clinical perspective the availability of pharmaceutical dosage forms such as stable solutions and lyophilizates of 5,10-CH₂-(6R)-THF having a high content of the active ingredient and being free of any kind of stabilizers would therefore be an advantage.

There thus still remains a great need for stable pharmaceutical dosage forms having a high content of 5,10-methylene-(6R)-tetrahydrofolic acid.

### SUMMARY OF THE INVENTION

It has now been found that surprisingly stable lyophilizates comprising 5,10-methylene-(6R)-tetrahydrofolic acid (5,10-CH₂-(6R)-THF) can be prepared from highly concentrated aqueous solutions of the sodium salt of 5,10-methylene-(6R)-tetrahydrofolic acid (5,10-CH₂-(6R)-THF*Na) which solutions further contain at least 40 mol-%, preferably from about 40 mol-% to 200 mol-%, even more preferred from about 50 mol-% to about 100 mol-% of an alkali metal sulfate, but no reducing agents or other stabilizing agents.

The lyophilizates of the present invention thus overcome the previously discussed known drawbacks and allow for the preparation of stable solid-state pharmaceutical compositions and dosage forms of high purity and a low content of either oxidation products or other chemical degradation products.

The advantageous stability and concentration characteristics of the lyophilizates of the present invention will allow the effective, and safe use in medicinal applications.

In a first aspect the present invention thus relates to a pharmaceutical dosage form which is a stable lyophilisate, which dosage form comprises 5,10-methylene-(6R)-tetrahydrofolic acid and does not contain stabilizers or any further chemotherapeutic agents.

A second aspect of the present invention is directed to a process for the preparation of the stable lyophilizates according to the first aspect, which process comprises the following steps:
i. dissolving (6S)-tetrahydrofolic acid in aqueous NaOH,
ii. adjusting the pH of the solution to 8.6±0.5,
iii. adding 100-120 mol % formaldehyde,
iv. stirring the reaction mixture until reaction has completed,
v. adding a solution of an alkali metal sulfate up to a final molar ratio of sulfate:5,10-methylene-(6R)-tetrahydrofolic acid from about 0.4:1 to about 1:2,
vi. filtering the reaction mixture to obtain a clear solution of 5,10-CH₂-(6R)-THF*Na, and
vii. freeze-drying the clear solution obtained in step vi.

In a third aspect the present invention further relates to a reconstituted solution of a pharmaceutical dosage form according to the first aspect, for use in the treatment of cancer, or in cancer therapy, in a human patient.

In a fourth aspect the present invention further relates to a method of treatment of cancer, or of cancer therapy, in human patients comprising administering a pharmaceutical dosage form according to the first aspect, or a reconstituted solution thereof, to a human patient in need thereof.

In a fifth aspect the present invention further relates to the use of a pharmaceutical dosage form according to the first aspect, or a reconstituted solution thereof, for the manufacture of a medicament for the treatment of cancer in human patients.

The lyophilizates of the present invention exhibit a stability over months or more without significant loss of active ingredient, e.g., maintaining the amount of active ingredient at or above 95% and more preferably at or above 98% for several months, including most preferably about 99%, 99.5% or 99.8%. This enables the manufacturing, storage and use of the lyophilizates of the present invention without significant decomposition before reconstitution.

### BRIEF DESCRIPTION OF THE FIGURES

FIG. 1 is adapted from Table 2 in WO 2007/064968 and demonstrates the stability over time of non-formulated and various formulated forms of 5,10-methylene-(6R,S)-tetrahydrofolic acid (% normalized purity). As can be seen, each formulation had a different stability profile. Thus, nonformulated 5,10-methylene-(6R,S)-tetrahydrofolic acid at neutral pH degraded rapidly over time. 24 hours following dissolution in water, the purity of nonformulated 5,10-methylene-(6R,S)-tetrahydrofolic acid was only 44.9% of the starting purity. The reference formulation formulated only with trisodium citrate (pH adjusted >7.5) showed slower degradation following dissolution in water.

However, purity after 24 hours was still only 65% compared to the starting purity, indicating degradation was not efficiently inhibited by the addition of trisodium citrate and adjustment of pH. The two test formulations #1 and #2 (i.e. 5,10-methylene-(6R,S)-tetrahydrofolic acid formulated with both ascorbic acid and trisodium citrate) were the most stable formulations (purity after 24 hours about 89%).

FIG. 2 is adapted from FIG. 1 in WO 2007/064968 and demonstrates graphically the tabulated results of FIG. 1 herein.

FIG. 3 is a table adapted from Example 1 of WO 2007/064968 showing the composition of the non-formulated and formulated forms of 5,10-methylene-(6R,S)-tetrahydrofolic acid shown in FIG. 1 and FIG. 2 herein.

FIG. 4 shows the purity analyses of four identical solutions of sodium salt of 5,10-methylene-(6R)-tetrahydrofolic acid of the present invention tested at four different conditions: 5° C. without a blanket of N₂, 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown for a total period of 7 hours. As can be seen from the graphs, the solutions are very stable under the storage conditions, changing from an initial purity between 96.6-97% to a purity of 96.4-96.5% (area %). As can also be seen, the effect of N₂ blanketing is minimal.

FIG. 5 shows analyses of the same four solutions of sodium salt of 5,10-methylene-(6R)-tetrahydrofolic acid as shown in FIG. 4 herein. In FIG. 5, the development over 7 hours of the main impurity, 10-formyl-(6R)-tetrahydrofolic acid (10-FTHFA) in the solutions as produced in Example 3 when stored at 2-8° C. is shown. As can be seen, the level of this impurity is practically constant over time.

FIG. 6 shows the long-term stability of a lyophilisate containing 5,10-methylene-(6R)-tetrahydrofolic acid at 5° C., 25° C. and at 40° C. As can be seen from the graphs, the lyophilisate are very stable, especially at lower temperatures such as 5° C., decreasing at 5° C. over 24 months to a purity of 99.3% resp. at 25° C. over 24 months to a purity of 96.6% (relative to the initial purity).

FIG. 7 shows analyses of the main impurity, 10-formyl-(6R)-tetrahydrofolic acid (10-FTHFA) for lyophilisate containing 5,10-methylene-(6R)-tetrahydrofolic acid at 5° C., 25° C. and at 40° C. As can be seen, the level of this impurity is practically constant over time.

### DEFINITIONS

As used herein, the term “sulfate” shall refer to an inorganic, aqueously soluble sulfate salt such as an alkali metal sulfate or alkali metal hydrogensulfate.

In the present text, the term “buffer” relates to citrate (or citric acid and salts thereof); dicarboxylates such as succinate, malate and maleate; tris(hydroxymethyl)aminomethane TRIS; N-tris(hydroxymethyl)methyl-2-aminoethanesulfonic acid (TES); 3-(N-morpholino) propanesulfonic acid (MOPS); N,N-bis(2-hydroxyethyl)-2-aminoethane-sulfonic acid (BES); MES; MOPSO; HEPES; phosphate; carbonate; ammonium; mono-, di-, and tri-alkylammonium; mono-, di-, and tri-hydroxylalkylammonium; glutamate; borate; lactate, as well as combinations of these.

In the present text, the term “reducing agent” relates to L-(+)ascorbic acid or salts thereof, reduced γ-glutathione, β-mercaptoethanol, thioglycerol and N-acetyl-L-cysteine.

In the present text, the term “solvent” relates to solvents which may be used in freeze drying processes. “Solutions” as referred to in the present text, comprise aqueous solutions as well as solutions in organic solvents. Typically, “aqueous solutions” mean solutions in water, saline solutions, water containing small amounts of buffers, water containing isotonic amounts of NaCl, or mixtures of water with organic solvents, and the like. Typical organic solvents include DMSO, acetonitrile, acetone, methanol, or ethanol.

### DETAILED DESCRIPTION OF THE INVENTION

It has as mentioned been found that surprisingly stable lyophilizates comprising 5,10-methylene-(6R)-tetrahydrofolic acid (5,10-CH₂-(6R)-THF) can be prepared from highly concentrated aqueous solutions of the sodium salt of 5,10-methylene-(6R)-tetrahydrofolic acid (5,10-CH₂-(6R)-THF*Na) which solutions further contain at least 40 mol-%, preferably from about 40 mol-% to 200 mol-%, even more preferred from about 50 mol-% to about 100 mol-% of an alkali metal sulfate, but no anti-oxidants or other stabilizing agents.

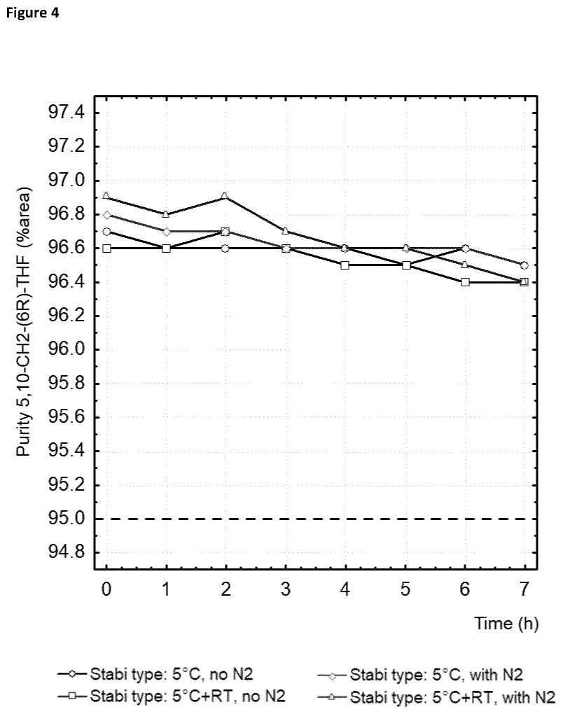

The highly concentrated solutions used for preparing the lyophilizates of the instant invention comprise 5,10-CH₂-(6R)-THF*Na and an alkali metal sulfate, as discussed above. These solutions have a high purity and are advantageous from a manufacturing perspective by remaining chemically stable for at least 7 hours at 5±3° C. or for at least 3 hours at room temperature, even without sparging the solution with nitrogen for minimizing degradation by oxidation (see FIG. 4). For example, the solution of 75 mg/ml is clear and remains clear regardless of whether it is stored at 2-8° C. or at RT, i.e., no precipitation occurs.

The pH of the solutions is typically in the range of 8.0 to 9.0, preferably in the range of 8.4 to 8.8 and can be adjusted during drug product manufacturing with e.g. small amounts of hydro-chloric acid or sodium hydroxide.

According to the present invention the highly concentrated solutions discussed hereinabove can be freeze-dried (lyophilized) to a stable, non-sticky powder and stored. The lyophilizate powder can be reconstituted with a diluent to a set concentration for administration. Such reconstituted lyophilizates can be administered either intramuscularly or intravenously.

Bulking agents such as mannitol may be added to the concentrated solution before the freeze-drying process to promote an acceptable lyophilized cake formation. Other excipients may be added, if required.

Also, electrolytes, sugars and/or polyols such as dextrose, glycerol, mannitol and sodium chloride may be added to adjust the osmolality. Osmolality adjustment can be done before (i.e. of the concentrated solutions) or after reconstitution of the lyophilizates of the present invention. The reconstituted lyophilisate solution preferably has an osmolality in the range of 250-350 mOsm. However, an osmolality of 200-600 mOsm can be tolerated as well and will depend on the volume to be administered as well as the injection/infusion time.

Lyophilization or freeze-drying is a dehydration process that works by freezing an aqueous solution containing a dissolved material therein and then reducing the surrounding pressure to allow the frozen water to sublimate directly from the solid phase to the gas phase. There are usually four stages in a complete lyophilization process: pretreatment, freezing, primary drying, and secondary drying.

Pretreatment includes any method of treating the material prior to freezing. This may include the addition of other components. Pretreatment is possible but not necessary in the preparation of stable lyophilizates of the present invention.

Freezing is often done by placing an aqueous solution of the material in a freeze-drying flask which is cooled by mechanical refrigeration, or by using dry ice or liquid nitrogen. On a larger scale, freezing the aqueous solution is usually done using a freeze-drying machine. In this step, it is important to cool the material below its triple point, the lowest temperature at which the solid and liquid phases of the material can coexist. This ensures that sublimation rather than melting will occur in the following steps. Freezing is preferably done at temperatures of −45° C. to −70° C. in the preparation of stable lyophilizates of the present invention.

Annealing for 1 to 2 hours at shelf temperatures around −5° C. to −2° C. is possible but not necessary in the preparation of stable lyophilizates of the present invention.

During the primary drying phase, the pressure is lowered (to the range of a few millibars), and enough heat is supplied to the material for the ice to sublimate. In this initial drying phase, about 95% of the water in the material is sublimated. This phase may be slow (can be several days in the industry), because, if too much heat is added, the material's structure could be altered. In the primary drying phase, pressure is controlled through the application of partial vacuum. The vacuum speeds up the sublimation, making it useful as a deliberate drying process. In the preparation of stable lyophilizates of the present invention, the primary drying phase is started at a freezing temperature of preferably between −45° C. to −70° C.

Then during the primary drying phase, the temperature is, after an optional starting period of preferably 10 minutes to 120 minutes at freezing temperature, increased over time to preferably about 0° C. During the primary drying phase, a pressure of preferably about 50 μbar to 200 μbar is maintained.

The secondary drying phase aims to remove unfrozen water molecules, since the ice was removed in the primary drying phase. In this phase, the temperature is raised higher than in the primary drying phase, and can even be above 0° C., to break any physico-chemical interactions that have formed between the water molecules and the frozen material. Usually, the pressure is also lowered in this stage to encourage desorption (typically in the range of microbars, or fractions of a pascal). Secondary drying is preferably done at temperatures up to about 25° C. to 30° C. and a pressure of about 50 μbar to 200 μbar in the preparation of stable lyophilizates of the present invention.

The primary and secondary drying phases may be combined by following a temperature ramp from freezing temperature to temperatures up to about 25° C. to 30° C. and a pressure ramp from about 50 μbar to 200 μbar in the preparation of stable lyophilizates of the present invention. The temperature ramp may contain multiple holding steps where the temperature is kept constant for some time. Preferably the holding steps, if any, are at freezing temperature, at about 0° C. and at about 25° C. to 30° C.

After the lyophilization process is complete, the vacuum is usually broken with an inert gas, such as nitrogen, before the material is sealed. At the end of the operation, the final residual water content of the lyophilizates of the present invention is usually below 5%, preferably at most 3%, even more preferably at most 2% and most preferably below 1%. In a particularly preferred embodiment, the lyophilizates of the present invention are essentially anhydrous.

Stability is a critical property and component of pharmaceutical formulation studies and drug development. Stability studies are performed both in solution and solid state. It is an established fact that the solution state and solid-state stability can differ both qualitatively and quantitatively. Extensive studies were performed for stability of the drug substance and pharmaceutical compositions thereof by exposing it to variety of stressors, like high temperature and/or high humidity. These studies also provide information on the degradation products and help in developing meaningful specifications as well as the intrinsic stability of the pharmaceutical composition. Most common pathways for drug degradation include i.a. hydrolysis, oxidation, and photochemical degradation.

The purpose of stability testing is to provide evidence on how the quality of a product varies with time under the influence of a variety of environmental factors such as temperature, humidity, and light, and to establish a suitable shelf life for the pharmaceutical product and recommended storage conditions, in order to ensure patient safety.

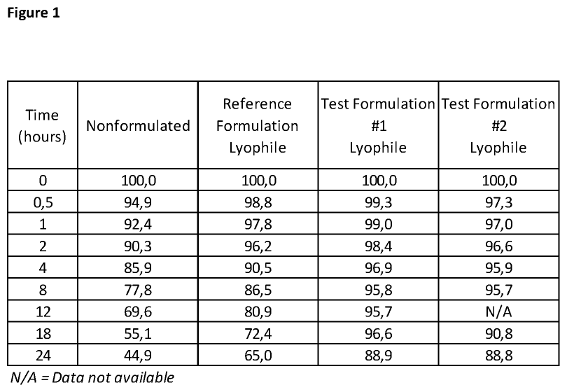

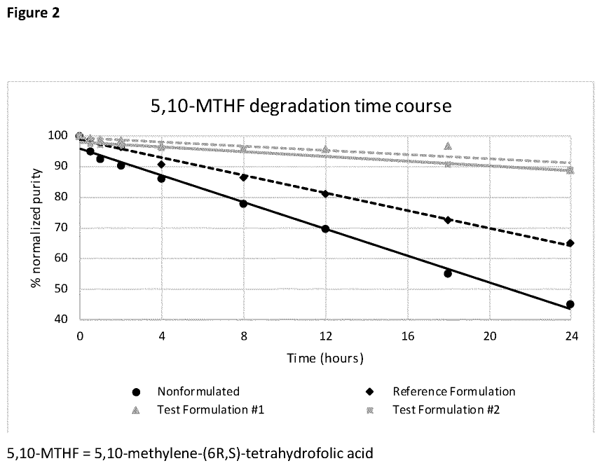

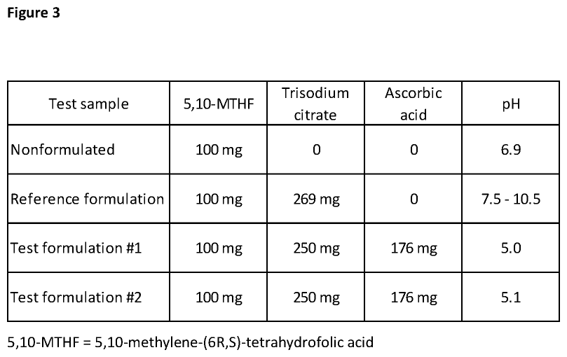

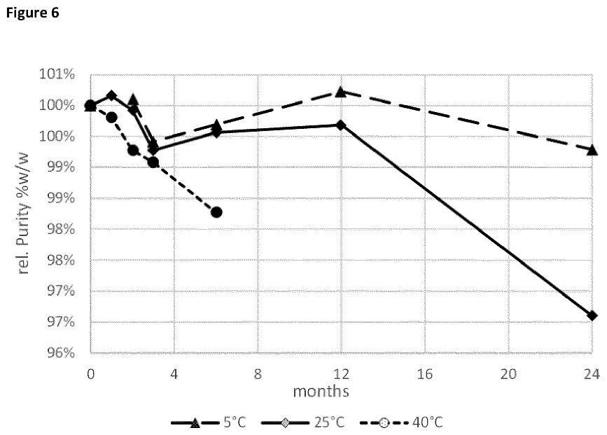

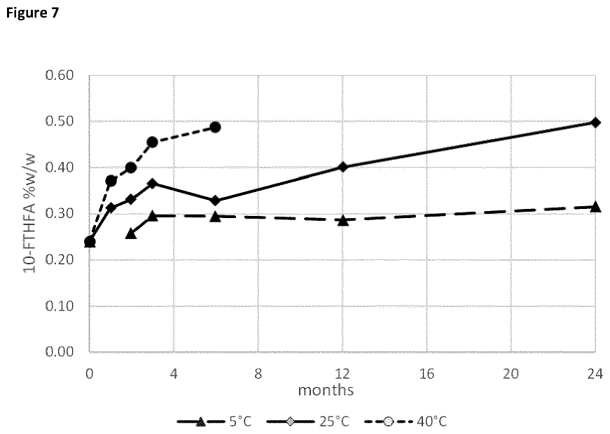

The high stability observed for the resulting lyophilizates of the present invention (see FIGS. 6 and 7) is highly surprising in view of the art described above, in which the presence of a stabilizer like citrate would have been mandatory. A comparison of FIG. 4 with FIGS. 1-3 thus strongly indicates that the high-content solutions used for the preparation of the lyophilizates of the present invention have similar or better stability than the ascorbate/citrate stabilized CoFactor® compositions discussed in i.a. WO 2007/064968.

In a first aspect the present invention thus relates to a pharmaceutical dosage form which is a stable lyophilisate, which dosage form comprises 5,10-methylene-(6R)-tetrahydrofolic acid and does not contain stabilizers or any further chemotherapeutic agents.

In a preferred embodiment the pharmaceutical dosage form of the present invention comprises the sodium salt of 5,10-CH₂-(6R)-tetrahydrofolic acid (5,10-CH₂-(6R)-THF*Na) and an alkali metal sulfate in a molar ratio from about 0.4:1 to about 1:2.

The present invention in one embodiment relates to a pharmaceutical dosage form according to the first aspect wherein the molar ratio of alkali metal sulfate:5,10-CH₂-(6R)-THF is from about 0.4:1 to about 1:2, preferably from about 0.5:1 to about 1:1.

Lyophilizates of the present invention are substantially amorphous while having an enhanced stability, such as an enhanced storage stability.

A second aspect of the present invention is directed to a process for the preparation of the stable lyophilizates according to the first aspect, which process comprises the following steps:
i. dissolving (6S)-tetrahydrofolic acid in aqueous NaOH,
ii. adjusting the pH of the solution to 8.6±0.5,
iii. adding 100-120 mol % formaldehyde,
iv. stirring the reaction mixture until reaction has completed,
v. adding a solution of an alkali metal sulfate up to a final molar ratio of sulfate:5,10-methylene-(6R)-tetrahydrofolic acid from about 0.4:1 to about 1:2,
vi. filtering the reaction mixture to obtain a clear solution of 5,10-CH₂-(6R)-THF*Na, and
vii. freeze-drying the clear solution obtained in step vi.

The reaction between (6S)-tetrahydrofolic acid and formaldehyde is quantitative, but it is advisable to employ a slight excess of formaldehyde to ensure that the reaction goes to completion. It should be avoided to employ too much formaldehyde, as this leads to increased levels of impurities (cf. Example 2a and 2b herein).

In a preferred embodiment of the third aspect, about 110 mol % formaldehyde is employed.

In another preferred embodiment, the alkali metal sulfate added in step v. is sodium sulfate.

Once the solution of 5,10-CH₂-(6R)-THF*Na has been generated, i.e. from step iv.-v., the temperature of the reaction mixture should be kept low, preferably around 0-5° C.

In an embodiment, the lyophilizate according to the first aspect may contain up to about 80% w/w 5,10-CH₂-(6R)-THF*Na.

In a third aspect the present invention further relates to a reconstituted solution of a pharmaceutical dosage form according to the first aspect, for use in the treatment of cancer, or in cancer therapy, in a human patient.

In a preferred embodiment, lyophilizates of the present invention are reconstituted into an aqueous pharmaceutical formulation to be administered into a patient in need thereof.

In a fourth aspect the present invention further relates to a method of treatment of cancer, or of cancer therapy, in human patients comprising administering a pharmaceutical dosage form according to the first aspect, or a reconstituted solution thereof, to a human patient in need thereof.

In a fifth aspect the present invention further relates to the use of a pharmaceutical dosage form according to the first aspect, or a reconstituted solution thereof, for the manufacture of a medicament for the treatment of cancer in human patients.

A further aspect is directed to a reconstituted solution of a pharmaceutical dosage form according to the first aspect which comprises 5,10-CH₂-(6R)-THF*Na, an alkali metal sulfate and a pharmaceutically acceptable carrier or diluent, such as sterile water or a liquid pharmaceutically acceptable vehicle, optionally further comprising at least one additional therapeutic agent including but not limited to, bactericides, antibiotics, antivirals, antiseptics, antineoplastics, anticancer compounds such as chemotherapeutic agents, antifungals, and/or anti-inflammatory agents or other bioactive or therapeutic agents that are suitable for human use, in particular anticancer compounds such as chemotherapeutic agents, for example 5-FU and derivatives, and antifolates, e.g. methotrexate, Pemetrexed.

### EXAMPLES

### HPLC

For the measurement of purity/content and degradation products an HPLC-UV Gradient Method was used: Column type: ODS, Mobile phase: A: aqueous Buffer; Mobile Phase: B: aqueous Buffer/Methanol, Run time: 30 min, Sample Solvent: aqueous Buffer.

### Water Content

The determination of the water content was performed according to Ph. Eur. 2.5.32/USP <921/Method Ic>.

### Osmolality

The determination of the osmolality was performed according to Ph. Eur. 2.2.35 (osmometer)/USP <785>.

### Example 1: Preparation of a Concentrated Aqueous Solution Comprising Sulfate and Sodium 5,10-methylene-(6R)-tetrahydrofolate

(a) 7.93 g (16 mmol) (6S)-tetrahydrofolic acid and 78.0 g distilled water were provided in a roundbottom flask at room temperature under N₂. The resulting suspension was stirred, and the pH adjusted to pH 11 by slow addition of a 32% NaOH solution. As soon as the solution became clear, a 1 M HCl solution was added gradually to adjust the pH of the reaction mixture to 8.3 at 25° C. The obtained clear solution was cooled to about 0° C., at which temperature it showed a pH of 8.8. The pH was again adjusted with 1 M HCl to pH=8.6 and 1.44 g of a 36.8% HCHO solution (110 mol %) was added in one portion. Upon completion of the addition the solution was stirred at 0° C. (ice bath) for 1 hour. Active charcoal (0.2 g, Norit C Extra) was added and the reaction mixture was stirred for 30 minutes at 0° C. and then cold filtered over a suction filter to obtain a clear solution of 5,10-CH₂-(6R)-THF*Na, which was used in step (b) without further purification.
(b) A chilled solution of 2.8 gr Na₂SO₄ (20 mmol, 1.25 mol %) in 15 ml distilled water was added to the solution as obtained in step (a). The pH was then adjusted with 1 M NaOH to 9.3±0.1, and the obtained reaction mixture was stirred under N₂ at 0° C. for 2 hours. Active charcoal (0.2 g, Norit C Extra) was added and the reaction mixture was stirred for 30 minutes at 0° C. and then cold filtered over a suction filter followed by sterile filtration through a 0.22 μm filter to obtain a clear solution of an approximately 1:1 molar composition of sodium 5,10-CH₂-(6R)-THF*Na and sodium sulfate. The solution contains about 8 gr 5,10-CH₂-(6R)-THF*Na per 100 ml, i.e. a concentration of about 80 mg/ml, corresponding to about 7.3 gr 5,10-CH₂-(6R)-THF free acid in 100 ml. The solution should be kept at 2-8° C.
(c) Cool the solution from step (b) to 2-8° C. and pass it through a 0.22 μm filter while keeping the solution as cold as possible. Fill the filtered solution into glass vials (2 ml or 160 mg 5,10-CH₂-(6R)-THF*Na per vial) while keeping the solution as cold as possible.

The influence of formaldehyde excess on product quality was analysed in the two following examples which were carried out identically except from the excess of formaldehyde. In example 2a, 110 mol % formaldehyde was used, whereas in example 2b 200 mol % formaldehyde was used. The use of 110 mol % formaldehyde in Example 2a provided the purest product.

### Example 2a: Preparation of a 5,10-methylene-(6R)-tetrahydrofolate Solution with Sulfate

4.72 g (6S)-Tetrahydrofolic acid were added under nitrogen to 220 ml water containing 10 g NaOH 2M (initial pH 13.74). The pH was kept at 9.3±0.1 until complete dissolution with 22.8 g NaOH 2M. Then 0.901 g of a 36.8% HCHO solution were added (110 mol %). The solution was stirred for 30 minutes. A chilled solution of 4.5 gr Na₂SO₄ (20 mmol, 1.25 mol %) in 15 ml distilled water was added to the solution and thereafter the pH was adjusted again to 9.3 with aqueous sodium hydroxide 2M (0.05 g). The so obtained solution contained 5,10-methylene-(6R)-tetrahydrofolic acid and sulfate with a purity of 94.8% area.

### Example 2b: Preparation of a 5,10-methylene-(6R)-tetrahydrofolate Solution with Sulfate

4.72 g (6S)-Tetrahydrofolic acid were added under nitrogen to 220 ml water containing 10 g NaOH 2M (initial pH 13.83). The pH was kept at 9.3±0.1 until complete dissolution with 22.8 g NaOH 2M. Then 1.639 g formaldehyde solution (36.76%) were added (200 mol %). The solution was stirred for 30 minutes. A chilled solution of 4.5 gr Na₂SO₄ (20 mmol, 1.25 mol %) in 15 ml distilled water was added to the solution and thereafter the pH was adjusted again to 9.3 with aqueous sodium hydroxide 2M. The so obtained solution contained 5,10-methylene-(6R)-tetrahydrofolic acid and sulfate with a purity of 91.5% area.

### Example 3: Preparation of a Stabilizer-Free Lyophilisate of a Concentrated Aqueous Solution

Fill the filtered solution from Example 1 at a temperature of 2-8° C. into vials (2 ml or 150 mg 5,10-CH₂-(6R)-THF per vial) while keeping the solution as cold as possible. Freeze-dry the vials and seal them under a slight vacuum with nitrogen in the headspace. Crimp the vials. The resulting lyophilisate contains 70-80% w/w 5,10-CH₂-(6R)-THF.

### Example 4: Stability Testing (Concentrated Solutions)

The solutions as produced in Example 1, step c, were tested for stability under four different conditions: 7 hrs at 5° C. without a blanket of N₂, 7 hrs at 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown in FIG. 4. As can be seen from the graphs, the solutions are very stable under the storage conditions, changing from an initial purity between 96.6-97% to a purity of 96.4-96.5% (area %). As can also be seen from FIG. 4, the effect of N₂ blanketing on stability is minimal.

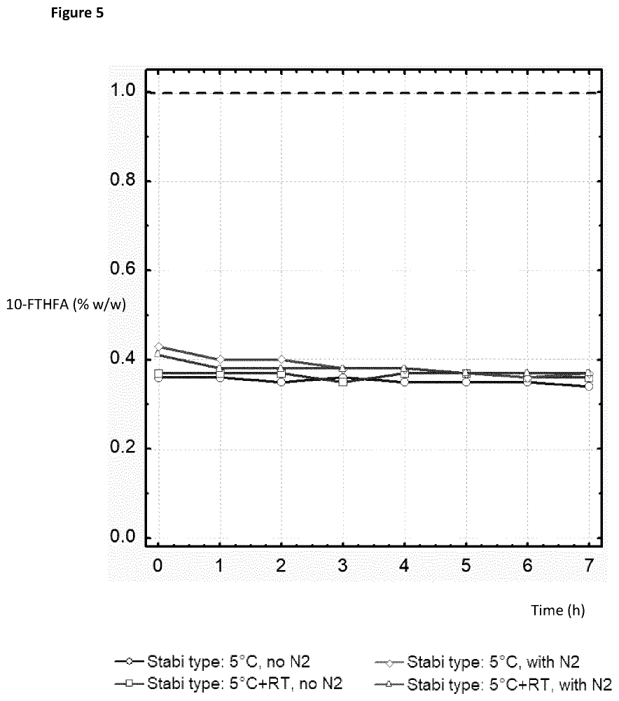

As part of the stability analysis, the development over 7 hours of the main impurity, 10-formyl-(6R)-tetrahydrofolic acid (10-FTHFA) in the solutions as produced in Example 1, step c when stored at 2-8° C. was also measured (see Table 1 below and FIG. 5). As can be seen, the level of this impurity is practically constant.

TABLE 1
Analysis of impurities in a solution comprising sodium 5,10-methylene-
(6R)-tetrahydrofolic acid and sulfate (main degradation product)

| | Content of 10-formyl-(6R)-tetrahydrofolic acid over | | | | | | | |
|---|---|---|---|---|---|---|---|---|
| Time (hrs) | storage time at 2-8° C. | | | | | | | |
| at 2-8° C. | 0 | 1 | 2 | 3 | 4 | 5 | 6 | 7 |
| (% w/w) | 0.36 | 0.36 | 0.35 | 0.36 | 0.35 | 0.35 | 0.35 | 0.34 |

### Example 5: Stability Testing (Lyophilizates)

In order to determine the long-term stabilities of lyophilisates of 5,10-CH₂-(6R)-THF prepared according to Example 3, lyophilisates were stored in air at +5° C., +25° C./60% relative humidity and +40° C./75% relative humidity. The content of 5,10-CH₂-(6R)-THF remaining was measured by HPLC at periodic intervals and is given by comparison with the initial value (% rel.). The results are shown in Table 2 and FIG. 6.

TABLE 2
Long-term stability of a lyophilisate containing
5,10-methylene-(6R)-tetrahydrofolic
acid as prepared according to Example 3

| | Relative content of (6R)-5,10-CH₂-THF | | | | | | |
|---|---|---|---|---|---|---|---|
| | over storage time [months] | | | | | | |
| | 0 | 1 | 2 | 3 | 6 | 12 | 24 |
| +5° C. | 100.0 | | 100.1 | 99.4 | 99.7 | 100.2 | 99.3 |
| +25° C./60% rh | 100.0 | 100.1 | 99.9 | 99.3 | 99.6 | 99.7 | 96.6 |
| +40° C./75% rh | 100.0 | 99.8 | 99.3 | 99.1 | 98.3 | | |

Table 2 and FIG. 6 clearly show that lyophilisates of 5,10-CH₂-(6R)-THF are highly stable over a long period of time, especially at lower temperatures such as 5° C.

In order to determine the long-term stabilities of lyophilisates of (6R)-5,10-CH₂-THF prepared according to Example 3, lyophilisates were stored in air at +5° C., +25° C./60% relative humidity and +40° C./75% relative humidity. The content of one of the main degradation product 10-formyltetrahydrofolic acid (10-FTHFA) was measured by HPLC at periodic intervals. The results are shown in Table 3 and FIG. 7.

TABLE 3
Long-term stability of a lyophilisate containing 5,10-methylene-
(6R)-tetrahydrofolic acid as prepared according to Example 3

| | Content of one of the main degradation | | | | | | |
|---|---|---|---|---|---|---|---|
| | product 10-formyltetrahydrofolic acid | | | | | | |
| | (10-FTHFA) over storage time [months] | | | | | | |
| | 0 | 1 | 2 | 3 | 6 | 12 | 24 |
| +5° C. | 0.24 | | 0.26 | 0.30 | 0.29 | 0.29 | 0.32 |
| +25° C./60% rh | 0.24 | 0.31 | 0.33 | 0.37 | 0.33 | 0.40 | 0.50 |
| +40° C./75% rh | 0.24 | 0.37 | 0.40 | 0.45 | 0.49 | | |

Table 3 and FIG. 7 confirm that lyophilisates of 5,10-CH₂-(6R)-THF are highly stable over a long period of time, especially at lower temperatures such as 5° C., also as witnessed by the very slow increase in content of one of the main degradation product 10-formyltetrahydrofolic acid (10-FTHFA).

1. A pharmaceutical dosage form which is a stable lyophilisate, which dosage form comprises 5,10-methylene-(6R)-tetrahydrofolic acid and does not contain stabilizers or any further chemotherapeutic agents.

2. The stable lyophilisate according to claim 1, wherein
said stabilizers are selected from the group consisting of citrate (or citric acid and salts thereof); tris(hydroxymethyl)aminomethane (TRIS); N-tris(hydroxymethyl) methyl-2-aminoethanesulfonic acid (TES); 3-(N-morpholino)propanesulfonic acid (MOPS); N,N-bis(2-hydroxy-ethyl)-2-aminoethanesulfonic acid (BES); MES; MOPSO; HEPES; phosphate; acetate; succinate; carbonate; ammonium; mono-, di-, and trialkylammonium; mono-, di-, and tri-hydroxylalkylammonium; maleate; glutamate; borate; lactate, L-(+)ascorbic acid or salts thereof; reduced γ-glutathione; β-mercaptoethanol; thioglycerol; N-acetyl-L-cysteine, and combinations of these.

3. The stable lyophilisate according to claim 1 which comprises a sodium salt of 5,10-methylene-(6R)-tetrahydrofolic acid and an alkali metal sulfate.

4. The stable lyophilisate according to claim 2, which consists of a sodium salt of 5,10-methylene-(6R)-tetrahydrofolic acid, sodium sulfate, water and optional osmolality correcting additives.

5. The stable lyophilisate according to claim 2 wherein a molar ratio of alkali metal sulfate:sodium salt of 5,10-methylene-(6R)-tetrahydrofolic acid is from 0.4:1.0 to 1:2.

6. The stable lyophilisate according to claim 1, with a concentration of 5,10-methylene-(6R)-tetrahydrofolic acid in the lyophilizate of at least 60% w/w.

7. The stable lyophilizate according to claim 6 having a water content below 5%.

8. The stable lyophilizate according to claim 7 which is anhydrous.

9. The stable lyophilizate according to claim 6 which contains 5,10-methylene-(6R)-tetrahydrofolic acid of a purity greater than 98%.

10. A reconstituted product obtained by dissolving or diluting the stable lyophilizate of claim 1 in water or a liquid pharmaceutically acceptable vehicle.

11. The reconstituted product according to claim 10, wherein the water is sterile water for injection.

12. The reconstituted product according to claim 10, further comprising a pharmaceutically acceptable carrier.

13. The reconstituted product according to claim 10, further comprising an additional pharmaceutically acceptable active ingredient.

14. The reconstituted product according to claim 10, further comprising a buffer, one or more osmolality correcting excipients, or both.

15. The reconstituted product according to claim 10 for use in the treatment of cancer or in cancer therapy.

16. The stable lyophilisate according to claim 1, with a concentration of 5,10-methylene-(6R)-tetrahydrofolic acid in the lyophilizate of at least 65% w/w.

17. The stable lyophilisate according to claim 1, with a concentration of 5,10-methylene-(6R)-tetrahydrofolic acid in the lyophilizate of between 70-75% w/w.

18. The stable lyophilizate according to claim 6 having a water content of at most 3%.

19. The stable lyophilizate according to claim 6 having a water content of at most 2%.

20. The stable lyophilizate according to claim 6 having a water content below 1%.
